(19) United States
(12) Patent Application Publication
(10) Pub. No.: US 2020/0245791 A1
(43) Pub. Date: Aug. 6, 2020

(54) Method for Adjusting the Height of Pillow and Pillow with Adjustable Height

(71) Applicant: Paodong (Xiamen) Technology Co., Ltd., Xiamen City (CN)

(72) Inventors: Xuan Zheng, Xiamen City (CN); Jiangping Miao, Xiamen City (CN); Jinghuang Kang, Xiamen City (CN); Yanjing Zhong, Xiamen City (CN); Diwei Wu, Xiamen City (CN)

(21) Appl. No.: 16/656,675

(22) Filed: Oct. 18, 2019

(30) Foreign Application Priority Data
Feb. 1, 2019 (CN) 201910102762.5

(51) Int. Cl. A47G 9/10; A61B 5/103

(52) U.S. Cl. CPC A47G 9/1027; A61B 5/1036

(57) ABSTRACT

The present invention provides a method for adjusting pillow height, which comprises the following steps: 1) Taking out the airbag from the pillow with adjustable height, applying a certain weight on the airbag, inflating and deflating the airbag, recording the airbag heights corresponding to the airbag under different air pressures, and obtaining the corresponding relations between the air pressures of the airbag and the heights; 2) During the process of adjusting the pillow height, the air pressure inside the airbag is read through a barometer, and the current height of the airbag is obtained through the corresponding relation between the air pressure and the height; 3) Stop inflating or deflating the airbag when the current height of the airbag is consistent with the target height. The present invention also provides a pillow for adjusting the height, which uses the method for adjusting the height of the pillow as described above.

### PRIORITY

This application claims the benefit of Chinese patent application serial No. 201910102762.5, entitled “Method for Adjusting the Height of Pillow and Pillow with Adjustable Height,” filed Feb. 1, 2019, herein incorporated by reference in its entirety.

### TECHNICAL FIELD

The invention relates to bedding, in particular to a pillow.

### BACKGROUND

With the general acceleration of people's life rhythm, lack of sleep has become a common phenomenon among urban people today. Experts remind that lack of sleep is very harmful to health and should be addressed seriously. The harm of insufficient sleep is mainly reflected in: 1. Affect the creative thinking of the brain; 2. Affect the growth and development of teenagers; 3. Affect people's health, etc. This shows that a comfortable sleep is very important to the human body. In bed rest, the head needs to be supported by a pillow and tilted upward at a certain angle. Therefore, the suitability of pillows is particularly important. If the pillow is not suitable, it will cause cervical vertebra pain, insomnia and dreaminess, and fatigue the next day, which will affect the normal life the next day. However, it is extremely difficult to choose a suitable pillow because of each person's body curve, sleeping posture and other factors.

Since the suitable pillow heights are different when people lie on their sides or supine, there are some pillows on the market that can automatically adjust the height according to different sleeping postures. The pillow achieves the purpose of adjusting the height of the pillow by inflating and deflating the airbag. During the adjustment process, the height of the airbag needs to be known to ensure that the pillow can reach the height value required by the user. As the airbag is a flexible device, it is difficult to detect the height of the pillow after inflation.

### SUMMARY

The main technical problem to be solved by the present invention is to provide a method for adjusting the height of a pillow, by which the height of the pillow is indirectly measured during the use of the pillow.

The present invention also provides a pillow with adjustable height, which uses the method for adjusting the height of the pillow as described above.

In order to solve the mentioned technical problems, the present invention provides a method for adjusting the height of a pillow, which comprises the following steps:

1) Taking out the airbag from the pillow with adjustable height, applying a certain weight on the airbag, inflating and deflating the airbag, recording the airbag heights corresponding to the airbag under different air pressures, and obtaining the corresponding relations between the air pressures of the airbag and the heights;

2) During the process of adjusting the pillow height, the air pressure inside the airbag is read by a barometer, and the current height of the airbag is obtained through the corresponding relation between the air pressure and the height;

3) When the current height of the airbag is consistent with the target height, inflation or deflation of the airbag is stopped.

In a preferred embodiment: In step 1, a ∩ shaped device is placed on the airbag so that the airbag is positioned in the ∩ shaped device, a certain weight is applied on the upper surface of the ∩ shaped device to simulate the pressure of the user's head on the pillow during sleeping, and the heights of the upper surface of the ∩ shaped device under different air pressures are read to obtain the heights of the airbag under different air pressures.

The present invention also provides a pillow with adjustable height, which uses the method for adjusting the height of the pillow as described above.

Being comparing with the prior art, the technical scheme of the present invention has the following beneficial effects:

According to the method for adjusting the height of the pillow provided by the present invention, the height of the airbag is corresponding to the air pressure of the airbag, so that the purpose of indirectly detecting the height of the airbag is achieved by detecting the air pressure of the airbag in the process of adjusting the pillow height and converting the air pressure into the height. This method has the advantages of small calculation task and high speed. When the pillow is raised or lowered to the target position, the inflation or deflation of the airbag can be quickly stopped, thus having strong practicability.

### BRIEF DESCRIPTION OF THE DRAWINGS

FIG. 1 is a method for adjusting pillow height.

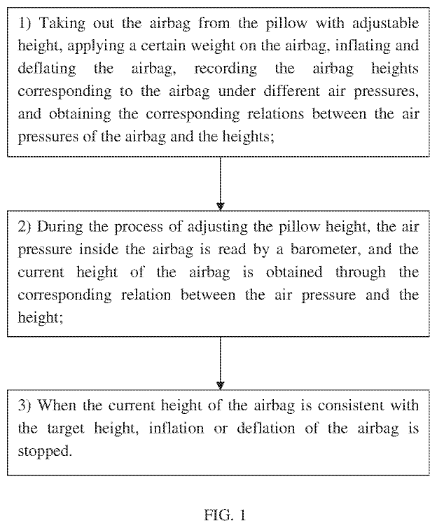

### DETAILED DESCRIPTION

The present invention will be further explained by specific examples.

The present invention provides a method for adjusting pillow height, which comprises the following steps:

1) Taking out the airbag from the pillow with adjustable height, applying a certain weight on the airbag, inflating and deflating the airbag, recording the airbag heights corresponding to the airbag under different air pressures, and obtaining the corresponding relations between the air pressures of the airbag and the heights;

Specifically, in this embodiment, the air pressures of the airbag are measured at 2 KPa, 3 KPa, 5 KPa and 8 KPa respectively, and the corresponding heights of the airbag are measured, and a weight is 3 kg to simulate the weight of a human head.

During measurement, a ∩ shaped device is placed on the airbag, so that the airbag is positioned in the ∩ shaped device, a weight of 3 kg is applied to the upper surface of the ∩ shaped device to simulate the pressure of the user's head on the pillow during sleeping, and the heights of the upper surface of the ∩ shaped device under different air pressures are read, so that the heights of the airbag under different air pressures are obtained. In order to ensure the accuracy of the measurement, four positions are selected on the upper surface of the ∩ shaped device to carry out the measurement, and the average value is finally taken.

The measured data are as follows:

| Air | Data 1 | Data 2 | Data 3 | Data 4 | |
|---|---|---|---|---|---|
| Pressure | (scale | (scale | (scale | (scale | |
| Value | reading) | reading) | reading) | reading) | Average |
| (Kpa) | (CM) | (CM) | (CM) | (CM) | (CM) |
| 2 | 17.5 | 17.5 | 17.7 | 17.9 | 17.65 |
| 3 | 18.2 | 18.4 | 18.5 | 18.5 | 18.4 |
| 5 | 19.6 | 19.6 | 19.6 | 19.6 | 19.6 |
| 8 | 20 | 20 | 20 | 20 | 20 |

2) During the process of adjusting the pillow height, the air pressure inside the airbag is read by a barometer, and the current height of the airbag is obtained through the corresponding relation between the air pressure and the height;

3) When the current height of the airbag is consistent with the target height, inflation or deflation of the airbag is stopped.

The embodiment also provides a pillow with adjustable height, which uses the method for adjusting the height of the pillow as described above.

The above is only the preferred embodiment of the present invention, but the scope of the present invention is not limited thereto. And those skilled in the art can easily think of the disclosed changes or technical scope of the present invention. Alternatives are intended to be included within the scope of the present invention. Therefore, the scope of protection of the present invention should be determined by the scope of the Claims.

1-3. (canceled)

4. A method for adjusting a height of a pillow, wherein the pillow includes an airbag, comprising:
taking out the airbag from the pillow;
applying a weight on the airbag to simulate a user's head on the pillow during sleeping;
inflating or deflating the airbag to obtain an air pressure inside the airbag, wherein a value of the air pressure is pre-selected;
measuring a height of the airbag, thereby acquiring a corresponding relation between the air pressure and the height;
repeating the inflating or deflating of the airbag and the measuring of the air pressure to vary the air pressure and the height, thereby acquiring a set of corresponding relations between the air pressure and the height; and
after acquiring the set of corresponding relations, adjusting the airbag to reach a target height by obtaining a corresponding air pressure inside the airbag.

5. The method of claim 4, wherein the inflating or deflating the airbag includes placing a ∩ shaped device on the airbag.

6. The method of claim 5, wherein the weight is applied on an upper surface of the ∩ shaped device.

7. The method of claim 5, wherein the height of the airbag is obtained by measuring a height of an upper surface of the ∩ shaped device.

8. The method of claim 4, wherein the adjusting of the airbag to reach the target height includes when the height of the airbag is consistent with the target height, stopping inflation or deflation of the airbag.

9. The method of claim 4, wherein the weight is 3 kilogram (Kg).

10. The method of claim 4, wherein the acquiring of the set of corresponding relations includes setting the air pressure to 2 Kpa, 3 Kpa, 5 Kpa, and 8 Kpa, respectively.

11. The method of claim 4, wherein the acquiring of the set of corresponding relations includes measuring the height multiple times for each air pressure to acquire an average height.

12. The method of claim 4, wherein the acquiring of the set of corresponding relations includes measuring the height four times for each air pressure to acquire an average height.

13. The method of claim 4, wherein the air pressure inside the airbag is measured by a barometer.

14. A method for adjusting a pillow with adjustable height by inflating or deflating an airbag in the pillow, comprising:
acquiring a set of corresponding relations between heights and air pressures of the airbag;
given a pre-determined height, acquiring a corresponding air pressure from the set of corresponding relations; and
inflating or deflating the airbag to obtain the corresponding air pressure, thereby reaching the pre-determined height.

15. The method of claim 14, wherein the inflating or deflating of the airbag to obtain the corresponding air pressure is monitored by a barometer.

16. The method of claim 14, wherein the acquiring of the set of corresponding relations includes:
taking out the airbag from the pillow;
applying a weight on the airbag; and
varying the air pressures inside the airbag and recording the corresponding heights.

17. The method of claim 16, wherein the weight is indirectly applied on the airbag.

18. The method of claim 17, wherein the weight is applied on a device with a ∩ shape, wherein the airbag is positioned in the device.

19. The method of claim 18, wherein the weight is applied on an upper surface of the ∩ shape.

20. The method of claim 18, wherein the corresponding heights are recorded by measuring heights at multiple locations in an upper surface of the ∩ shape.

21. A pillow with adjustable height, comprising:
an airbag; and
a barometer, wherein adjusting a height of the pillow includes:
acquiring a table of corresponding relations between a plurality of heights and a plurality of air pressures inside the airbag through a series of measurements; and
inflating or deflating the airbag to reach the height by obtaining an air pressure corresponding to the height from the table of corresponding relations, wherein obtaining the air pressure is monitored by the barometer.

22. The pillow of claim 21, wherein the series of measurements includes for each of the plurality of air pressures, measuring corresponding heights for multiple times to get a corresponding average height.

23. The pillow of claim 22, wherein the measuring of the corresponding heights for multiple times is performed on multiple locations of an upper surface of a device positioned above the airbag.
